(19) United States
(12) Patent Application Publication
(10) Pub. No.: US 2005/0124963 A1
(43) Pub. Date: Jun. 9, 2005

(54) Method for treating aspects of alcoholism, addiction and psychosis

(76) Inventor: Philip Seeman, Toronto (CA)

Correspondence Address: BERESKIN AND PARR, 40 KING STREET WEST, BOX 401, TORONTO, ON, M5H 3Y2, CA

(21) Appl. No.: 10/718,517

(22) Filed: Nov. 24, 2003

(51) Int. Cl. A61B 19/00

(52) U.S. Cl. 604/407

(57) ABSTRACT

This application relates to a method for treating various aspects of alcoholism, including alcohol craving and relapse into alcoholism, by the administration of an effective amount of gaseous anaesthesia to a deep surgical anaesthesia. The method can be applied for the alleviation of addiction and craving associated with amphetamine use, and for the alleviation of psychosis in schizophrenia and the alleviation of psychosis in Alzheimer's disease. In addition, the state of addiction or the state of psychosis, or the state of alcohol withdrawal can be monitored in animal tissue in vitro or in humans in vivo by measuring the density of the high-affinity states of the dopamine D2 receptors in the striatum or other brains regions containing these receptors which are elevated in these conditions.

### FIELD OF THE INVENTION

The present invention relates to methods for treating aspects of alcoholism, including the reduction in craving for alcohol or amphetamine, diminishing psychosis in medical illnesses, and diminishing or preventing relapse into alcoholism.

### BACKGROUND OF THE INVENTION

Alcohol addiction and relapse is a major cause of disease and death. Although ethanol releases dopamine in the brain, little is known about factors persisting after ethanol withdrawal which may precipitate relapse. Although ethanol enhances the electrically-stimulated release of dopamine (Nestby et al., Psychopharmacology 133: 69-76, 1997), and enhances the action of amphetamine (Manley & Little, J. Pharmacol. Exper. Therap. 281: 1330-1339, 1997), there are no significant changes or increases in type 2 dopamine receptors (also known as D2 receptors) in the brain, despite their critical and central role in psychomotor behaviour (Volkow et al., Psychiat. Res.: Neuroimaging 116, 163-172, 2002). Although the dopamine D2 receptor can exist in either a high-affinity state or a low-affinity state for dopamine, it is the high-affinity state, D2^{High}, which is the physiologically functional state, because the concentrations of dopamine agonists which inhibit the release of prolactin are identical to their dissociation constants at the high-affinity state of D2 (George et al., Endocrinology 117: 690-697, 1985). The dissociation constant, K, of dopamine at the high-affinity state, D2^{High}, is 1.75 nM.

Without an increase in dopamine type 2 receptors, it has been puzzling how an increase in dopamine sensitivity occurs in ethanol withdrawal (Balldin et al., Psychopharmacology 86: 142-146, 1985; Hannigan et al., Neurotoxicol. Teratol. 12: 79-84, 1990; Schmidt et al., Prog. Neuropsychopharmacol. Biol. Psychiat. 26: 209-217, 2002; Robinson & Berridge, Addiction 95 [Supplement 2]: S91-S117, 2000). Alternative mechanisms to explain the dopamine supersensitivity require an ethanol challenge or stimulation of the tissue or animal. A similar paradox in amphetamine-induced supersensitivity was resolved (Seeman et al., Synapse 46: 235-239, 2002) by finding an increase in the functional form of D2, namely the D2 high-affinity state, or D2^{High}.

There is a need for the understanding of the dopamine sensitivity that occurs upon ethanol withdrawal. Such an understanding will lead to treatments for alcohol addiction and relapse,

### SUMMARY OF THE INVENTION

The present inventor searched for changes in D2^{High} in the striata of rats withdrawn from ethanol. These sites were found to be persistently elevated for two weeks, corresponding to the relapse period of several months in humans. One hour of deep general anesthesia with isoflurane restored D2^{High} to normal.

Accordingly, the present invention relates to a method of determining if a test subject is in ethanol withdrawal comprising measuring an amount of high-affinity dopamine D2 receptors in striata of the test subject. In a further embodiment, if the amount of the high-affinity dopamine D2 receptors in striata of the subject is greater than that for a control subject, then the test subject is in ethanol withdrawal.

The present invention also relates to a method for monitoring a test subject for susceptibility to relapse from ethanol withdrawal comprising measuring an amount of high-affinity dopamine D2 receptors in striata of the test subject. The amount of high-affinity dopamine D2 receptors in striata of the test subject is correlated with the susceptibility of the test subject to relapsing from ethanol withdrawal. In embodiments of the invention, if the amount of high-affinity dopamine D2 receptors in striata of the test subject is greater than that for a control subject, then the test subject is less susceptible to relapse from ethanol withdrawal. In further embodiments, the amount of high-affinity dopamine D2 receptors in striata of the test subject is monitored over a period of time and when said amount begins to decrease, the test subject is more susceptible to relapse from ethanol withdrawal.

In another aspect of the present invention, there is provided a method for reducing elevated amounts of high-affinity D2 receptors which occur upon ethanol withdrawal, which method comprises administering, to subjects in need thereof, an effective amount of a general gaseous anaesthesia.

Further, the present invention relates to a method of treating a condition associated with an elevated amount of high-affinity sites of dopamine D2 receptors comprising administering an effective amount of a general gaseous anaesthesia to subjects in need thereof. In embodiments of the invention the condition is selected from one or more of craving for alcohol, relapse from ethanol withdrawal, medical illness associated with an elevated density of high-affinity dopamine D2 receptors and addiction and craving associated with amphetamine use. In a further embodiment the medical illness associated with an elevated density of high-affinity dopamine D2 receptors is psychosis, for example psychosis associated with schizophrenia and/or Alzheimer's disease.

The present invention also relates to the use of general gaseous anaesthesia to reduce elevated amounts of high-affinity sites of D2 which occur and persist upon withdrawal of ethanol. Further, the invention relates to a use of general gaseous deep surgical anaesthesia to treat a condition associated with an elevated amount of high-affinity sites of dopamine D2 receptors.

In a further embodiment of the invention, the amount of high-affinity dopamine D2 receptors in the striatum is used to determine the state of dopamine sensitivity of subjects who abstain from ethanol drinking.

### DETAILED DESCRIPTION OF THE INVENTION

The present inventor has found that in rats there is a 360% elevation in the amount of D2^{High} upon withdrawal of ethanol. Further the inventor has found that these sites remain elevated for almost two weeks before returning to normal. One hour of deep general anesthesia with isoflurane restored D2^{High} to normal levels.

Accordingly, the present invention relates to a method of determining if a test subject is in ethanol withdrawal comprising measuring an amount of high-affinity dopamine D2 receptors in striata of the test subject. In a further embodiment, if the amount of high-affinity dopamine D2 receptors in striata of the test subject is greater than that for a control subject, then the subject is in ethanol withdrawal.

The term “amount of high-affinity dopamine D2 receptors” in general refers to any quantitative measure of the number of sites or states of high-affinity dopamine D2 receptors in a specified area. In an embodiment of the invention the term “amount” refers to the density of the sites or states of high-affinity dopamine D2 receptors. The term “density” refers to a measure of quantity per unit volume.

The term “test subject” as used herein refers to any mammal, including humans, suspected of being in alcohol withdrawal. In an embodiment the test subject is human.

The term “control subject” as used herein refers to any mammal, including humans, which is not in alcohol withdrawal or does not suffer any other condition that would result in elevated levels of high-affinity dopamine D2 receptors. In an embodiment the control subject is the same species as the test subject. In an embodiment of the invention, the amount of high affinity dopamine D2 receptors in the striata of the control subject is about 0.5-3 pmol/g.

The term “about” as used herein means within experimental error.

The term “greater than” as used herein in reference to the amount of high-affinity dopamine D2 receptors in striata of a test subject compared to that for a control subject means that the amount of high-affinity dopamine D2 receptors in the test subject is at least about 30%, specifically at least about 100%, above that that in the control subject.

As used herein, the terms “alcohol” and “ethanol” are used interchangeably. Alcohol refers to any liquid or beverage comprising ethanol.

The term “striatum” (pl. striata) as used herein refers to the brain region corresponding to the combined region of the putamen and the caudate nucleus in the human brain, and also, more generally, refers to any brain region which contains an appreciable amount or density of dopamine D2 receptors such as the substantia nigra, the ventral tegmental area, the olfactory tubercles, the limbic pathway, the cingulate cerebral cortex, the nucleus accumbens and the cerebral cortex.

The present invention also relates to a method for monitoring a test subject for susceptibility to relapse from ethanol withdrawal comprising measuring an amount of high-affinity dopamine D2 receptors in striata of the test subject. The amount of high-affinity dopamine D2 receptors in striata of the test subject is correlated with the susceptibility of the test subject to relapsing from ethanol withdrawal. In embodiments of the invention, if the amount of high-affinity dopamine D2 receptors in striata of the test subject is greater than that for a control subject, then the test subject is less susceptible to relapse from ethanol withdrawal. In further embodiments, the amount of high-affinity dopamine D2 receptors in striata of the test subject is monitored over a period of time and when said amount begins to decrease, the test subject is more susceptible to relapse from ethanol withdrawal.

In an embodiment of the invention, the amount of high-affinity dopamine D2 receptors in the striatum is used to determine the state of dopamine sensitivity of subjects who abstain from ethanol drinking. Accordingly, the present invention also relates to a method for determining the state of dopamine sensitivity of a test subject who abstains from ethanol drinking comprising measuring an amount of high-affinity dopamine D2 receptors in striata of the test subject. The amount of high-affinity dopamine D2 receptors in striata of the test subject is correlated with the state of dopamine sensitivity of subjects who abstain from ethanol drinking. In embodiments of the invention, if the amount of high-affinity dopamine D2 receptors in striata of the test subject is greater than that for a control subject, then the test subject is in an enhanced state of dopamine sensitivity.

The amount of high-affinity dopamine D2 receptors may be determined using a method which specifically and directly measures the density of D2^{High}, but which does not use the method of measuring the proportion of high- and low-affinity states by means of [³H]antagonist competition with dopamine. In an embodiment of the invention, the amount is determined using an in vitro method which comprises determining a difference in the density of [³H]raclopride sites in the presence and absence of guanilylimidodiphosphate, the difference being the density of high-affinity sites for D2. This method is described in the examples herein below and in Seeman, et al., Synapse 46: 235-239, 2002. The amount of high-affinity dopamine D2 receptors may also be determined by determining an amount of [¹¹C](+)-4-propyl-9-hydroxynaphthoxazine ([¹¹C](+)PHNO) bound after intravenous injections of high and low specific activity, and using the amount bound in the cerebellum to define non-specific binding as described in the examples herein below and in Seeman et al., Synapse 14: 254-262, 1993 and Seeman et al., Synapse 46: 235-239, 2002. In an embodiment of the invention, the amount of ([¹¹C](+)PHNO bound is determined using positron emission tomography (PET). In general, this in vivo method to quantitate the density of the high-affinity state of brain dopamine D2 receptors in vivo, involves: 1. A dose of [¹¹C](+)PHNO with high specific activity is injected intravenously in a subject; the striatum is scanned for gamma ray emission, while the cerebellum serves to define the level of nonspecific radioactivity. 2. About 6 hours later, a dose of [¹¹C](+)PHNO with low specific activity is injected intravenously in the subject; the striatum and cerebellum are scanned once more. 3. The amount of [¹¹C](+)PHNO bound to the striatum (in pmol/ml striatum) for the two doses are calculated and graphed as a two-point Scatchard plot, where the amount of specifically bound [¹¹C](+)PHNO (in pmoles/ml striatum, corrected for nonspecific binding in the cerebellum) is graphed on the abscissa, and the amount of specifically bound [¹¹C](+)PHNO (pmoles/ml) divided by the nonspecific binding in the cerebellum (picomoles/ml) is plotted on the ordinate. Extrapolation to the abscissa yields the density of the high-affinity states of the dopamine D2 receptors.

Another in vitro method to determine the amount of high-affinity dopamine D2 sites involves the use of radio-domperidone which is readily sensitive to dopamine concentrations of 1-100 nM, corresponding to the high-affinity state of the D2 receptor (Seeman et al., Synapse 49: 209-215, 2003). Radio-domperidone is far more sensitive to displacement by low concentrations of dopamine, compared to radio-raclopride or radio-spiperone, both of which are not significantly displaced by 1-100 nM dopamine. While radio-domperidone is ideal for monitoring the high-affinity state of D2 in vitro, it cannot be used in vivo because of its very low ability to permeate the blood-brain barrier.

If the amount of high-affinity D2 receptors is found to be greater in the test subject than in the control subject using any one of the above-mentioned methods, then the test subject is in ethanol withdrawal.

In another aspect of the present invention, there is provided a method for reducing elevated amounts of high-affinity D2 receptors which occur upon ethanol withdrawal, which method comprises administering, to subjects in need thereof, an effective amount of a general gaseous anaesthesia.

Further, the present invention relates to a method of treating a condition associated with an elevated amount of high-affinity sites of dopamine D2 receptors comprising administering an effective amount of a general gaseous anaesthesia to subjects in need thereof. In embodiments of the invention the condition is selected from one or more of craving for alcohol, relapse from ethanol withdrawal, medical illness associated with an elevated density of high-affinity dopamine D2 receptors and addiction and craving associated with amphetamine use. In a further embodiment the medical illness associated with an elevated density of high-affinity dopamine D2 receptors is psychosis, for example psychosis associated with schizophrenia and/or Alzheimer's disease.

The present invention also relates to the use of general gaseous anaesthesia to reduce elevated amounts of high-affinity sites of D2 which occur and persist upon withdrawal of ethanol. Further, the invention relates to a use of general gaseous deep surgical anaesthesia to treat a condition associated with an elevated amount of high-affinity sites of dopamine D2 receptors.

As used herein the term “elevated amounts” means that the amount of high affinity dopamine D2 receptors in a test subject is at least about 30%, specifically at least about 100%, above that found in control subjects.

The term an “effective amount” or a “sufficient amount” of an agent as used herein is that amount sufficient to effect beneficial or desired results, including clinical results, and, as such, an “effective amount” depends upon the context in which it is being applied. For example, in the context of administering an agent that reduces elevated amounts of high-affinity sites of D2, an effective amount of an agent is, for example, an amount sufficient to achieve such a reduction of elevated density of high-affinity sites of D2 as compared to the response obtained without administration of the agent.

To “suppress” or “reduce” a function or activity, such as elevated amount of high-affinity dopamine D2 receptors, is to reduce the function or activity when compared to otherwise same conditions except for a condition or parameter of interest, or alternatively, as compared to another conditions. In an embodiment of the invention, the term “reduce the elevated amount of high affinity dopamine D2 receptors” means to reverse this elevated amount to near normal levels. By “normal levels” it means levels that are present in the absence of alcohol withdrawal or any other conditions that would result in increased amounts of high-affinity dopamine D2 receptors.

As used herein, and as well understood in the art, “treatment” is an approach for obtaining beneficial or desired results, including clinical results. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, and remission (whether partial or total), whether detectable or undetectable. “Treatment” can also mean prolonging survival as compared to expected survival if not receiving treatment.

“Palliating” a disease or disorder means that the extent and/or undesirable clinical manifestations of a disorder or a disease state are lessened and/or time course of the progression is slowed or lengthened, as compared to not treating the disorder.

In an embodiment of the invention, the general gaseous anaesthesia is administered to the subjects more than once. In further embodiments of the invention, the anaesthesia is general gaseous deep surgical anaesthesia. In further embodiments of the invention the anaesthesia is selected from one or more of isoflurane, halothane, diethyl ether, enflurane, methoxyflurane, ethyl chloride, vinyl ether, fluroxene, cyclopropane, ethylene, chloroform, trichloroethylene, high concentrations of nitrous oxide, and similar general anesthetic gaseous agents. In further embodiments, the anaesthesia is selected from one or more of isoflurane and halothane.

The following non-limiting examples are illustrative of the present invention:

### EXAMPLES

Methods

To induce ethanol dependence, rats (male Sprague Dawley, 200 g) received an intraperitoneal injection of 0.9% NaCl or 2 g/kg ethanol twice daily (i.e., 1.4 ml of 18% ethanol [in 0.9% NaCl] per 100 g at 9 AM and again at 3 PM daily) for 10 days (Lindholm et al., Alcohol 22: 165-171, 2000). On different days during the withdrawal period, each rat underwent CO₂ narcosis and cervical dislocation; the striata were removed. The frozen (−70° C.) striata were blotted and weighed. The striatal D2 receptors (with or without guanilylimidodiphosphate) were measured (Scatchard) by [³H]raclopride, as reported elsewhere (Seeman et al., Synapse 46: 235-239, 2002). The binding of [³H]PHNO (or [+]-4-propyl-9-hydroxynaphthoxazine) (0.1 to 4.5 nM; 114 C/mmol; custom synthesized by New England Nuclear Corp.; non-specific binding defined by 10 μM S-(−)-sulpiride) to striatal D2 receptors was done as reported elsewhere (Seeman et al., Synapse 14: 254-262, 1993; Synapse 46: 235-239, 2002).

Results

Using [³H]raclopride, the density of D2 receptors in saline-treated rat striata were found to be 20.9±0.7 pmol/g (pmols per g of original striatum) (Kd, 1.39±0.08 nM; n=27). In the presence of 200 μM guanilylimidodiphosphate, the density of D2 in the control tissues increased by 2.0 pmol/g to 22.9±0.68 pmol/g (Kd, 1.55±0.14 nM). Therefore, the normal density of high-affinity states of the type 2 dopamine receptor is 2.0 pmol/g.

During the first eight days after stopping ethanol treatment, the ethanol-treated striata show a D2 density of 19±0.8 pmol/g (Kd, 1.45±0.15 nM; n=8). In the presence of guanilylimidodiphosphate, the receptor density increases by 7.2±0.6 pmol/g to 26.1±0.8 pmol/g (Kd, 2±0.2 nM)(FIG. 1), an increase of 360% over the control D2^{High} value of 2 pmol/g.

The elevated level of D2^{High} sites comes down to 4.8 pmol/g on days 11 and 12 after stopping ethanol, and is back to normal (2 pmol/g or less) on day 15.

To confirm the elevation of D2^{High} sites by another method, [³H]PHNO (or [+]-4-propyl-9-hydroxynaphthoxazine), a selective D2 agonist (Seeman et al., Synapse 14: 254-262, 1993) can be used. Using [³H]PHNO, the striatal D2 density is 8.6 pmol/g (Kd, 0.71 nM), falling to 6 pmol/g with guanilylimidodiphosphate, indicating that the control D2^{High} is 2.6 pmol/g.

Using [³H]PHNO, D2^{High} for the ethanol-treated striata (7 days withdrawal) is 7.1 pmol/g, an increase of 273% over the control value of 2.6 pmol/g, in approximate agreement with the above data using [³H]raclopride.

Ethanol-treated rats (five days into withdrawal) were anesthetized with isoflurane for one hour (deep stage 3 surgical anesthesia). Twenty-four hours later, at which time the high-affinity states fully recover (Seeman & Kapur, Synapse 50: 35-40, 2003), the rat striata revealed a normal D2^{High} density of 1.3 pmol/g.

Discussion

The data show that upon withdrawal of ethanol there is a 360% elevation of the density of D2^{High}, and that these sites remain elevated for almost two weeks before returning to normal. One hour of deep general anesthesia with isoflurane restored D2^{High} to normal.

The sites resistant to the action of guanine nucleotide represent the low-affinity sites of D2 labelled by either [³H]raclopride or [³H]PHNO.

The absolute concentration of D2^{High} has not been generally measured by others using the present methods. Although it is customary to determine the proportion of high-affinity states of G-linked receptors by the competition between an antagonist radioligand and an exogenously-added agonist, this method generally does not reveal a significant difference in the proportion of high-affinity states between control striata and drug-treated striata.

The advantage of the present method, using guanine nucleotide and [³H]raclopride saturation curves, is that it yields the density of the high-affinity states without altering the endogenous concentration of the agonist dopamine.

The 3.2-fold increase in D2^{High} would readily contribute to the increased sensitivity of the ethanol-withdrawn individual to amphetamine and dopamine agonists. For example, an increase of 40% in functional D2 receptors would readily account for a five-fold leftward shift in the agonist dose-response curve (List & Seeman, Adv. Biochem. Psychopharmacol. 24: 95-101, 1980). Hence, an elevation of 360% would markedly shift leftward the behavioral sensitivity to amphetamine or apomorphine.

Finally, because the D2^{High} states are the functional form of D2, their increase may explain the enhanced dopamine sensitivity of animals and humans in alcohol withdrawal and may contribute to ethanol relapse in humans, as found with lisuride (Schmidt et al., Prog. Neuropsychopharmacol. Biol. Psychiat 26: 209-217, 2002). It is known that the risk of alcohol relapse in alcoholics is highest during the first four months after alcohol detoxification. The present data in the rats reveal an elevation in D2^{High} sites which persists for two weeks after stopping ethanol administration, a period of time equivalent to several months in humans.

Although it is not known whether such a persistent elevation of D2^{High} may contribute to alcohol relapse or may on the other hand prevent relapse in humans, this issue can now be examined in humans by positron emission tomography using [¹¹C](+)PHNO. It is here proposed that as long as D2 sites are elevated, the mood of the individual remains elevated and the risk of relapse is relatively low. It is further proposed that the time when the elevated D2^{High} sites begin to return to normal is the period associated with relapse.

Because deep surgical anesthesia rapidly restores a normal D2^{High} level, it is proposed that deep general anesthesia with a gaseous anesthetic, such as isoflurane or halothane or close congeners of such gaseous anesthetics, reduces the craving or eliminate the tendency by humans to relapse into alcoholism. This general principle and method can be applied to other medical illnesses associated with an elevation in the level or density of the high-affinity states of dopamine D2 receptors, including amphetamine use and abuse, with or without psychosis, and psychosis associated with schizophrenia, and psychosis associated with Alzheimer's disease. In particular, post-mortem human tissues reveal that D2 receptors are elevated by 6% to 31% in Alzheimer's disease, and by 10% to 105% in schizophrenia (Seeman et al., Neuropsychopharmacology 1: 5-15, 1987). Because the high-affinity states of D2 are invariably a significant component of the total D2 population of sites, these D2 elevations indicate that these diseases have a considerable elevation in the density of high-affinity states of D2. Such elevations in the D2^{High} states would also be amenable to being lowered by treatment with the method of gaseous anesthesia, as described. In the case of amphetamine use, abuse and addiction, direct measurement of the high-affinity states of D2 in animals (rats) sensitized to amphetamine has found these sites to be elevated by over 350%, an elevation of which, and addiction of which, would be readily diminished by the method of gaseous anesthesia, as described herein.

Because of this central psychomotor role for the high-affinity state of D2, it is beneficial to have a method which selectively measures the high-affinity state of the dopamine D2 receptor in the human brain in vivo.

As will be apparent to those skilled in the art in the light of the foregoing disclosure, many alterations and modifications are possible in the practice of the invention without departing from the spirit or scope thereof. Accordingly, the scope of the invention is to be construed in accordance with the substance defined by the following claims.

All publications, patents and patent applications are herein incorporated by reference in their entirety to the same extent as if each individual publication, patent or patent application was specifically and individually indicated to be incorporated by reference in its entirety. Where a term in the present application is found to be defined differently in a document incorporated herein by reference, the definition provided herein is to serve as the definition for the term.

1. A method of determining if a test subject is in ethanol withdrawal comprising measuring an amount of high-affinity dopamine D2 receptors in striata of the test subject.

2. The method according to claim 1, wherein if the amount of high-affinity dopamine D2 receptors in striata of the test subject is greater than that for a control subject, then the test subject is in ethanol withdrawal.

3. The method according to claim 1, wherein the amount of high-affinity dopamine D2 receptors is the density of the sites or states of high-affinity dopamine D2 receptors.

4. The method according to claim 1, wherein the test subject is a human suspected of being in alcohol withdrawal.

5. The method according to claim 2, wherein the control subject is a human which is not in alcohol withdrawal or does not suffer any other condition that would result in elevated levels of high-affinity dopamine D2 receptors.

6. The method according to claim 2, wherein if the amount of high-affinity dopamine D2 receptors in the test subject is at least about 30% more that that in the control subject then the test subject is in ethanol withdrawal.

7. A method for monitoring a test subject for susceptibility to relapse from ethanol withdrawal comprising measuring an amount of high-affinity dopamine D2 receptors in striata of the test subject.

8. The method according to claim 7, wherein if the amount of high-affinity dopamine D2 receptors in striata of the test subject is greater than that for a control subject, then the test subject is less susceptible to relapse from ethanol withdrawal.

9. The method according to claim 8, wherein if the amount of high-affinity dopamine D2 receptors in the test subject is at least about 30% more that that in the control subject then the test subject is less susceptible to relapse from ethanol withdrawal.

10. The method according to claim 8, wherein the amount of high-affinity dopamine D2 receptors in striata of the test subject is monitored over a period of time and when said amount begins to decrease, the test subject is more susceptible to relapse from ethanol withdrawal.

11. A method for determining the state of dopamine sensitivity of a test subject who abstains from ethanol drinking comprising measuring an amount of high-affinity dopamine D2 receptors in striata of the test subject.

12. The method according to claim 11, wherein if the amount of high-affinity dopamine D2 receptors in striata of the test subject is greater than that for a control subject, then the test subject is in an enhanced state of dopamine sensitivity.

13. The method according to claim 12, wherein if the amount of high-affinity dopamine D2 receptors in the test subject is at least about 30% more that that in the control subject then the test subject is in an enhanced state of dopamine sensitivity.

14. A method for reducing elevated amounts of high-affinity D2 receptors which occur upon ethanol withdrawal, which method comprises administering, to subjects in need thereof, an effective amount of a general gaseous anaesthesia.

15. A method of treating a condition associated with an elevated amount of high-affinity sites of dopamine D2 receptors comprising administering an effective amount of a general gaseous anaesthesia to subjects in need thereof.

16. The method according to claim 15, wherein the condition is selected from one or more of craving for alcohol, relapse from ethanol withdrawal, medical illness associated with an elevated density of high-affinity dopamine D2 receptors and addiction and craving associated with amphetamine use.

17. The method according to claim 16, wherein the medical illness associated with an elevated density of high-affinity dopamine D2 receptors is psychosis.

18. The method according to claim 17, wherein the psychosis is associated with schizophrenia and/or Alzheimer's disease.

19. The method according to claim 15, wherein the general gaseous anaesthesia is administered to the subjects more than once.

20. The method according to claim 15, wherein the anaesthesia is general gaseous deep surgical anaesthesia.

21. The method according to claim 20, wherein the anaesthesia is selected from one or more of isoflurane, halothane, diethyl ether, enflurane, methoxyflurane, ethyl chloride, vinyl ether, fluroxene, cyclopropane, ethylene, chloroform, trichloroethylene, high concentrations of nitrous oxide, and similar general anesthetic gaseous agents.
